(19) United States
(12) Patent Application Publication
(10) Pub. No.: US 2014/0243846 A1
(43) Pub. Date: Aug. 28, 2014

(54) Balloon Catheter and Method of Use Thereof

(71) Applicant: COOK MEDICAL TECHNOLOGIES LLC, BLOOMINGTON, IN (US)

(72) Inventors: Steen Aggerholm, Heddinge (DK); Thomas Lysgaard, Solroed Strand (DK)

(73) Assignee: COOK MEDICAL TECHNOLOGIES LLC, BLOOMINGTON, IN (US)

(21) Appl. No.: 14/178,519

(22) Filed: Feb. 12, 2014

Related U.S. Application Data
(60) Provisional application No. 61/769,322, filed on Feb. 26, 2013.

(51) Int. Cl. A61B 17/22

(52) U.S. Cl. CPC A61B 17/22032
USPC 606/127

(57) ABSTRACT

One aspect of the invention provides a balloon catheter having a distal tip that does not extend distally beyond the distal end of the catheter balloon when the catheter balloon is inflated. In one embodiment, the distal end of the catheter balloon has a substantially flat profile when inflated. Other aspects of the invention provide methods of manufacturing and of using the balloon catheter. In one embodiment, the balloon catheter is used in the treatment of kidney stones.

### RELATED APPLICATIONS

The present patent application claims the benefit of the filing date under 35 U.S.C. §119(e) of U.S. Provisional Patent Application Ser. No. 61/769,322, filed Feb. 26, 2013, the contents of which are hereby incorporated by reference.

### TECHNICAL FIELD

The present invention relates generally to medical devices and more particularly to balloon catheters used to treat narrowed or obstructed portions of a body vessel. Certain aspects of the invention relate to methods of manufacturing and using such devices.

### BACKGROUND

Balloon catheters are widely used in the medical profession for various intraluminal procedures. One common procedure relates to the dilation of an obstructed portion of a body vessel. For example, certain body ducts that transport fluids are subject to obstruction by solid masses, or “stones”, formed from crystals that separate from the transported fluid and build up within the duct. Examples of such masses include renal stones, gall stones and gastric stones. In many instances, such masses pass out of the body without the need for intervention by a physician. However, stones that cause lasting symptoms or other complications require intervention to remove the stones from the body.

Renal stones are one of the most painful of urologic disorders. Such stones form within the kidney from crystals that separate from urine. Sometimes, such stones travel down the urinary tract and are expelled from the body. In other cases, a stone may cause a blockage in the urinary tract.

Narrow tubes called ureters carry urine from the kidneys to the bladder. The bladder stores urine and eventually empties the urine into the urethra, from which it is expelled from the body. Renal stones may form in the ureters and can contain various combinations of chemicals. One type of stone contains calcium in combination with either oxalate or phosphate. Another type of stone is formed from uric acid.

Gallstones are hard masses that form in the gallbladder, a sac-like organ under the liver on the right side of the abdomen. Such masses can obstruct the bile duct, a narrow tube connecting the gallbladder to the small intestine.

Extracorporeal shock wave lithotripsy (“ESWL”) is a minimally invasive treatment for the treatment of renal and gallbladder stones. In ESWL, ultrasonic sound waves that are created outside the body travel through the skin and body tissues until they hit the denser stones. The stones break down into smaller particles that can sometimes be expelled naturally from the body. Renal stones can also be removed using laser lithotripsy. This technique involves the insertion of a probe into the renal track. A cystoscope or ureteroscope is inserted into the patient's urethra, either directly or over a guide wire, and is advanced up the urinary tract to locate the target renal stone. Once the stone is located, a thin fiberoptic is introduced into a channel of the endoscope and advanced until it comes into contact with the stone. Light from a laser, for example, a holmium laser, is directed through the fiberoptic and the stone disintegrates or fragments.

In some cases, the above methods are not effective and surgery is the preferred treatment for the removal of such stones. A balloon catheter is typically used for opening the ureteral opening directly beneath the stone's location before patients undergo surgical treatment. Typically, a balloon catheter is inserted into the vessel and advanced to the occluded region. The balloon is then dilated by delivering a dilation fluid through a lumen present in the catheter shaft. Inflation of the balloon causes the exterior surface of the balloon to press against the wall of the body vessel and to expand the vessel.

The success of this procedure depends on the ability to position the inflatable balloon close to the stone blocking the vessel. However, many designs of balloon catheter include a tip portion positioned distally of the inflatable balloon. The presence of the tip limits the ability to position the balloon close to or against the stone. The use of a balloon catheter without such a tip portion offers a means of overcoming this problem. However, manufacturing balloon catheters without a tip portion presents several problems related to their complexity of manufacture.

### SUMMARY

In one aspect, the present invention provides a balloon catheter including an inflatable balloon and a catheter shaft. The distal end of the inflatable balloon attaches to the distal end of the catheter and the proximal end of the inflatable balloon attaches to the catheter shaft at a distance from the distal end of the inflatable balloon that is less than the unfolded longitudinal dimension of the inflatable balloon.

In one embodiment, the balloon is compressed in a longitudinal direction when deflated and, upon inflation, the distal region of the balloon extends distally beyond the distal end of the catheter. In another embodiment, the catheter shaft includes an inflation lumen extending from its proximal end towards the distal end to an inflation port in fluid contact with the interior of the balloon. In yet another embodiment, the catheter shaft includes a second lumen, which is sized to accept a wire guide.

In certain embodiments, the distal end of the balloon has a generally flat surface perpendicular to a longitudinal axis of the inflatable balloon. In other embodiments, the inflatable balloon has a rounded proximal end. The balloon can include a nylon, polyolefin, polyester, polyurethane, fluoropolymer, polyethylene, polytetrafluoroethylene, latex, rubber or mixtures of these materials.

Another aspect of the present invention provides a method of manufacturing a balloon catheter. In one embodiment, the method includes the steps of inserting a distal end of a catheter shaft into a proximal end of an inflatable balloon and advancing the distal end of the catheter shaft towards the distal end of the inflatable balloon to a position proximal of the distal end to the inflatable balloon. The proximal end of the inflatable balloon is bonded to the catheter shaft while maintaining the distal end of the catheter shaft at the position proximal of the distal end to the inflatable balloon. The distal end of the inflatable balloon is the moved proximally towards the distal end of the catheter shaft and is bonded at or near the distal end of the catheter shaft, so that the middle region of the inflatable balloon is compressed along a distal-proximal axis.

In one embodiment, the compression is such that, when the inflatable balloon is inflated, a distal region of the inflatable balloon moves distally to at least the distal end of the catheter shaft. In another embodiment, the distal region of the inflatable balloon moves distally beyond the distal end of the catheter shaft.

Yet another aspect of the present invention provides a method for treating an occluded or narrowed vessel. In one embodiment the method includes the step of positioning a distal end of a catheter shaft of a balloon catheter as described herein against the occluded or narrowed portion of the vessel and inflating the inflatable balloon, whereby a distal region of the inflatable balloon moves distally to at least the distal end of the catheter shaft and presses against the occluded portion of the vessel.

In one embodiment, the vessel is a vessel of the urinary track. The occlusion can be an occlusion resulting from the presence of a kidney stone.

### BRIEF DESCRIPTION OF THE DRAWINGS

FIG. 1 is a partial side view depicting one embodiment of a balloon catheter having the balloon in a deflated condition.

FIG. 2 is a partial side view of the balloon catheter shown in FIG. 1 with the balloon in an inflated condition.

FIGS. 3A-C illustrate one embodiment of a method of manufacturing the balloon catheter illustrated in FIGS. 1 and 2.

FIG. 4 is a flow diagram illustrating the method of manufacture shown in FIGS. 3A-C.

FIGS. 5A-C illustrate a partial side view of three embodiments of a balloon catheter with the balloon in an inflated condition. FIG. 5A illustrates an embodiment in which the proximal and distal ends of the balloon have a flattened profile. FIG. 5B illustrates an embodiment in which the distal end of the balloon has a flattened profile while the proximal end of the balloon has a rounded or substantially conical profile. FIG. 5C illustrates an embodiment in which the proximal and distal ends of the balloon have a rounded or substantially conical profile.

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of and “consisting essentially of,” the embodiments or elements presented herein, whether explicitly set forth or not.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

As used in the specification, the terms “proximal” and “distal” should be understood as being in the terms of a physician using the device. The term distal means the portion of the device which is farthest from the physician and the term proximal means the portion of the device which is nearest to the physician.

As used herein, the term “body vessel” means any body passage or lumen, including but not limited to vascular coronary or peripheral vessels, esophageal, intestinal, biliary, urethral and ureteral passages.

### Tipless Balloon Catheters

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

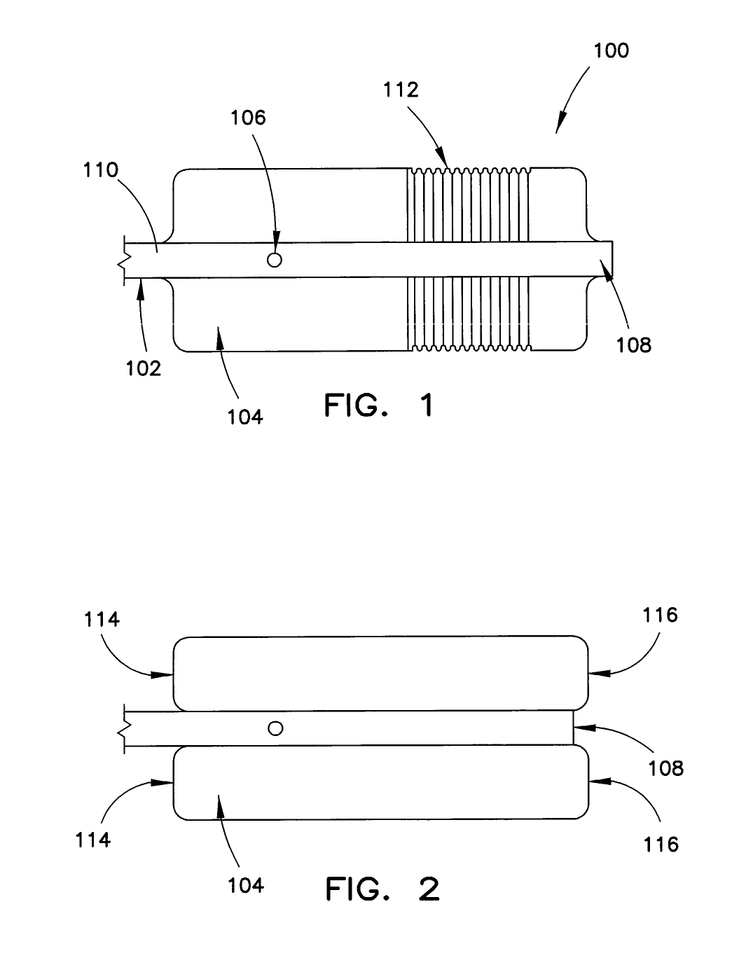

One aspect of the present invention provides balloon catheters having an inflatable balloon that extends distally to at least the distal end of the catheter shaft when the balloon is inflated (tipless balloon catheters.) Referring now to FIG. 1, one embodiment of the present invention provides an implantable balloon catheter 100 including catheter shaft 102, only the distal portion of which is illustrated, and catheter balloon 104 mounted at the distal end of catheter shaft 102. Catheter shaft 102 includes an internal lumen (not shown) extending from its proximal end to inflation port 106. Inflation port 106 is in fluid contact with the interior of catheter balloon 104 and provides a means of inflating catheter balloon 104. In certain embodiments, catheter shaft may also include a wire guide lumen (not shown) for use in the delivery of the balloon catheter. FIG. 1 shows catheter balloon 104 in a deflated configuration. Proximal end of catheter balloon 104 attaches to catheter shaft at position 110 and distal end of balloon 104 attaches to catheter shaft 102 at or near its distal end 108. The distance between attachment positions 108 and 110 is less than the length of catheter balloon 104. As a consequence, when deflated, catheter balloon 104 includes folded portions 112, formed as a result of the longitudinal compression of catheter balloon 102.

FIG. 2 illustrates balloon catheter 100 with catheter balloon 104 in an inflated condition. As catheter balloon 104 is inflated the ends of the balloon extend outwards. In particular, distal end 116 of catheter balloon 104 extends distally beyond the distal end 108 of catheter shaft 102 while proximal end 114 of catheter balloon 104 extends proximally. As a consequence, folded portions 112 straighten until catheter balloon 104 is in a fully inflated configuration. When catheter balloon 104 is fully inflated, the distal end 116 of catheter balloon is positioned at or distally of the distal end 108 of catheter shaft 102. In this configuration, the distal end 108 of catheter shaft 102 does not obstruct the positioning of catheter balloon directly against an obstruction in a body vessel.

Such a balloon may be formed of any flexible material, such as a nylon, polyolefin, polyester, polyurethane, fluoropolymer, polyethylene, polytetrafluoroethylene (PTFE), latex, rubber, and mixtures of these materials. In one embodiment, the thickness of the wall of the balloon is approximately 0.0005 inch thick. However, the wall of the balloon can be of any appropriate thickness provided that the thickness does not compromise properties that are important for achieving optimum performance. In various embodiments, the balloon wall thickness is within the range of approximately 0.0005 inch to 0.0012 inch thick.

In one embodiment, the balloon made from a low or non-compliant material, such as for example, nylon or polyester. A low or non-compliant balloon will increase in diameter by up to a maximum of about 5% of its normal diameter in response to increasing the pressure for inflating the balloon to between about 5 to 50 atmospheres. Alternatively, the balloon may be made from a hybrid or highly compliant material where the diameter of the balloon may increase as much as about 40% during inflation. The hybrid or highly compliant balloon may proportionally increase in diameter in response to increases in inflation pressure which may allow for fewer balloon sizes to be used to treat a wider range of vessel diameters.

In various embodiments, the rated burst pressure (RBP) of the balloon is equal to or greater than 10, 15, 20, 25, 30, 35, 40, 45 or 50 atmospheres. RBP is the statistically-determined maximum pressure to which a balloon may be inflated without rupturing. There is a 95% confidence that 99.9% of the balloons will not burst at or below the RBP upon single inflation. The balloon can be a reinforced balloon. For example, the balloon can be reinforced by incorporating a woven tubular textile sleeve over the balloon as disclosed in co-pending U.S. Publication Number 2011/0046654 (to Kuppurathanam) the content of which is incorporated by reference. In other embodiments, a reinforcing member is wound around the balloon. At least one other reinforcing member may also be wound around the balloon at an angle that crosses the first reinforcing member. The reinforcement can be attached to the balloon with an adhesive.

In certain embodiments, the catheter shaft of the balloon catheters disclosed herein includes one or more radiopaque markers to assist in the positioning of the balloon within the body vessel of a patient. In other embodiments, the balloon is inflated with an inflation fluid including a contrast medium.

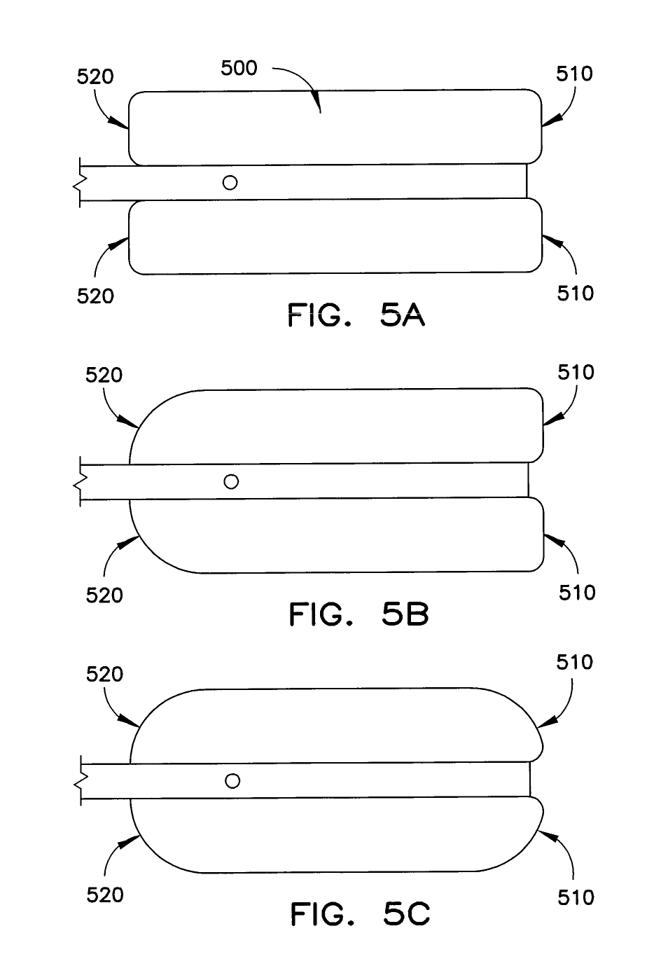

FIGS. 5A-C illustrate three alternative embodiments of the distal portion of a tipless balloon catheter. These embodiments differ in the shape of the distal and proximal end portions of the catheter balloon. In the embodiment illustrated in FIG. 5A, both the distal end 510 and the proximal end 520 of the balloon 500 have a flattened profile. In this embodiment, the ends of the inflatable balloon have a generally flat surface perpendicular to a longitudinal (proximal-distal) axis of the inflatable balloon. FIG. 5B illustrates an embodiment in which the distal end 510 of the catheter balloon has a flattened profile while the proximal end 520 of the balloon has a rounded or substantially conical profile. FIG. 5C illustrates an embodiment in which the proximal end 520 and distal end 510 of the balloon have a rounded or substantially conical profile.

In certain embodiments, the distal end of the catheter balloon is shaped such that, when fully inflated, at least 70, 75, 80, 85, 90, 97, 98 or 99 percentage of the distal surface of the catheter balloon is positioned perpendicular to the longitudinal axis of the catheter balloon such that, when the inflated balloon is positioned against a flat surface perpendicular to the longitudinal axis of the balloon catheter, the percentages of the distal surface of the catheter balloon listed above are in contact with the flat surface.

While the embodiment illustrated in FIGS. 1 and 5 may include a wire guide lumen that extends along the entire length of the catheter from the proximal end to the distal end, it is understood that the present embodiments may also include alternative lumen configurations. For example, the wire guide lumen may extend along only a portion of the length of the catheter. Indeed, over-the-wire, rapid exchange, short wire and intraductal exchange type delivery systems are contemplated and considered to be within the scope of the invention. A rapid exchange balloon catheter includes a proximal port for the wire guide lumen that is not located at or near the proximal end of the catheter shaft. It is instead located at an intermediate location along the catheter shaft. Examples of such alternative lumen configurations are illustrated in U.S. Publication Numbers 2011/0160834 (to Aggerholm) and 2009/0149808 (to Hansen and Aggerholm), the content of which are incorporated by reference.

### Methods of Manufacturing a Balloon Catheter

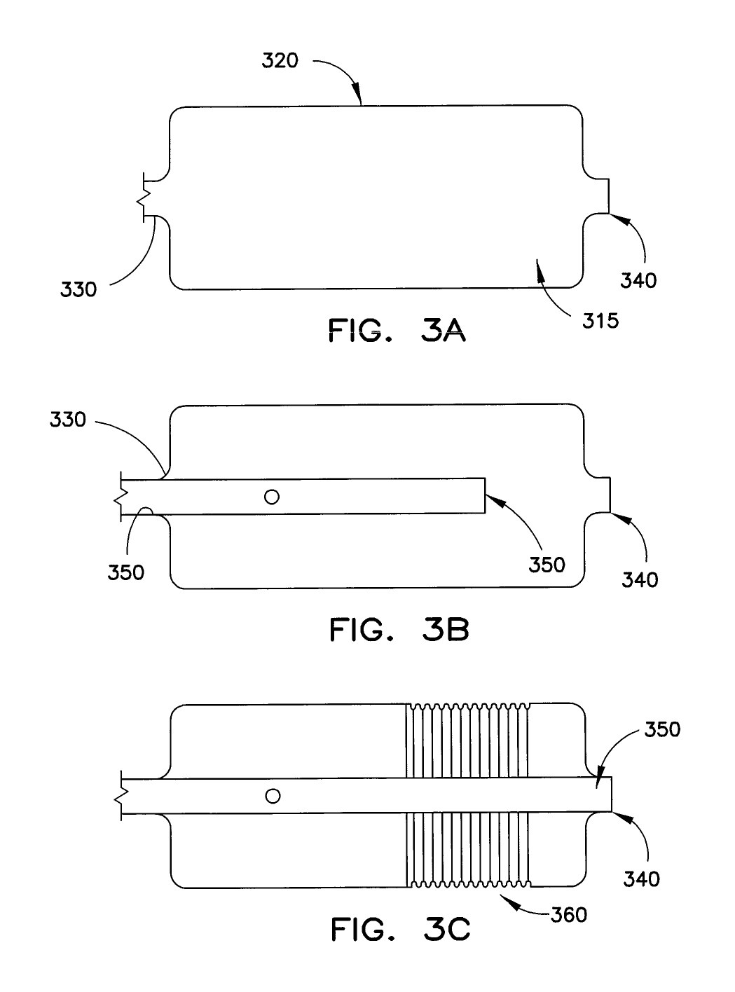

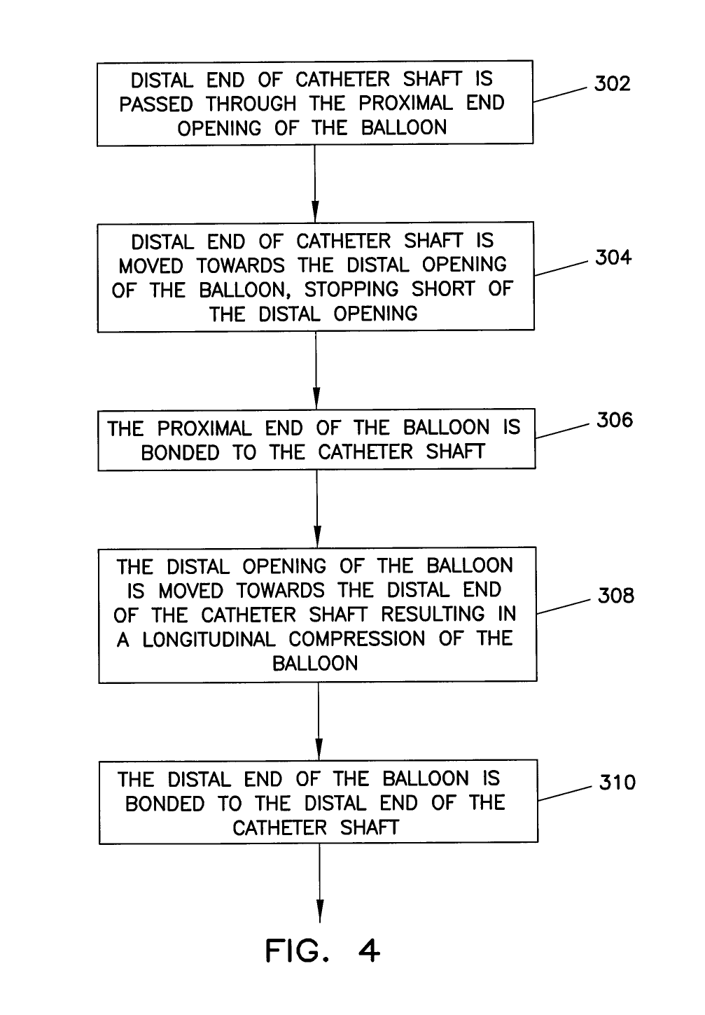

Another aspect of the present invention provides methods of manufacturing a tipless balloon catheter. FIGS. 3A-C and the flow diagram shown in FIG. 4 illustrate one such method of manufacturing such a balloon catheter. FIG. 3A illustrates an inflatable balloon 315 having a proximal end opening 330, a distal end opening 340 and a center portion 320. In one embodiment, the balloon is manufactured using a blow molding process including heating a material, such as a resin, within a mold to produce the balloon. The material may be heated via heaters or viscous dissipation, e.g., working of the material through the molding process, or any other suitable means for heating the material. Any polymer suitable for blow molding may also be used.

Turning now to FIG. 3B, balloon catheter shaft 350 is inserted through proximal opening 350 and advanced so that its distal end 350 is positioned proximally of distal opening 340. The proximal opening 330 of balloon 315 is then sealed to catheter shaft 350. The balloon may be attached by being hot melted, adhesively bonded or solvent fused to the catheter shaft 350. Any other suitable means known to those skilled in the art may also be used to attach the balloon to the shaft.

After attaching the proximal end of balloon 315 to the catheter shaft, the distal end 350 of balloon 315 is moved proximally to engage the distal end 340 of catheter shaft 350 and then sealed to the catheter shaft using one of the sealing methods described above. FIG. 3C shows the balloon catheter after this step is accomplished. The balloon is shown to include folded region 360, which accommodate the excess longitudinal dimension of the balloon.

### Methods of Treating an Occluded Body Vessel

Another aspect of the present invention provides a method for treating an occluded vessel using one of the embodiments of the balloon catheter disclosed herein. In one embodiment, the method includes the steps of positioning a distal end of the catheter shaft of a balloon catheter at an occluded or narrowed portion of the vessel and inflating the inflatable balloon by pumping saline or another inflation medium into the interior of the balloon. Upon inflation, the distal region of the inflatable balloon moves distally against the occluded region of the vessel and pushes against the occlusion and also against the vessel wall immediately adjacent to the obstruction.

In one embodiment, the vessel is a vessel of the urinary system. In such an embodiment, the occlusion may result from the presence of a kidney stone. For example, the balloon catheter may be used for opening the ureteral vessel before patients undergo kidney stone surgery. The absence of a distal tip portion allows the balloon to be positioned directly beneath the kidney stone's location. As a result of this positioning, the ureteral vessel may be opened directly below the location of the stone.

In another embodiment, the vessel is the bile duct. Gallstones are hard masses that form in the gallbladder, a sac-like organ under the liver on the right side of the abdomen. Such masses can obstruct the bile duct, a narrow tube connecting the gallbladder to the small intestine.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. A balloon catheter comprising:
an inflatable balloon having an unfolded longitudinal dimension and comprising a proximal end, a middle region and a distal end, and
a catheter shaft having a distal end,
wherein the distal end of the inflatable balloon attaches to the distal end of the catheter and wherein the proximal end of the inflatable balloon attaches to the catheter shaft at a distance from the distal end of the inflatable balloon that is less than the unfolded longitudinal dimension of the inflatable balloon.

2. The balloon catheter of claim 1, wherein the balloon is compressed in a longitudinal direction when deflated.

3. The balloon catheter of claim 1, wherein a distal region of the balloon extends distally beyond the distal end of the catheter upon inflation.

4. The balloon catheter of claim 1, wherein the catheter shaft comprises an inflation lumen extending from its proximal end towards the distal end and in fluid contact with an interior of the inflatable balloon.

5. The balloon catheter of claim 4, wherein the catheter shaft further comprises a second lumen, wherein the second lumen is sized to accept a wire guide.

6. The balloon catheter of claim 1, wherein the distal end of the inflatable balloon comprises a generally flat surface perpendicular to a longitudinal axis of the inflatable balloon.

7. The balloon catheter of claim 1, wherein the inflatable balloon comprises a rounded proximal end.

8. The balloon catheter of claim 1, wherein the catheter shaft comprises a radiopaque marker.

9. The balloon catheter of claim 1, wherein the inflatable balloon comprises a material selected from the group consisting of a nylon, polyolefin, polyester, polyurethane, fluoropolymer, polyethylene, polytetrafluoroethylene, latex, rubber, and mixtures thereof.

10. The balloon catheter of claim 1, wherein the inflatable balloon comprises polyethylene.

11. The balloon catheter of claim 1, wherein the inflatable balloon is a non-compliant balloon

12. A method of manufacturing a balloon catheter comprising:
inserting a distal end of a catheter shaft into a proximal end of an inflatable balloon, wherein the inflatable balloon comprises the proximal end, a middle region and a distal end,
advancing the distal end of the catheter shaft towards the distal end of the inflatable balloon to a position proximal of the distal end to the inflatable balloon,
bonding the proximal end of the inflatable balloon to the catheter shaft while maintaining the distal end of the catheter shaft at the position proximal of the distal end to the inflatable balloon, and
bonding the distal end of the inflatable balloon to the distal end of the catheter shaft, whereby the middle region of the inflatable balloon is compressed along a distal-proximal axis.

13. The method of claim 12, wherein the compression is such that, when the inflatable balloon is inflated, a distal region of the inflatable balloon moves distally to at least the distal end of the catheter shaft.

14. The method of claim 13, wherein the distal region of the inflatable balloon moves distally beyond the distal end of the catheter shaft.

15. A method for treating an occluded vessel comprising:
positioning a distal end of a catheter shaft of a balloon catheter against an occluded portion of the vessel, wherein the balloon catheter comprises:
an inflatable balloon having an unfolded longitudinal dimension and comprising a proximal end, a middle region and a distal end, and
a catheter shaft having a distal end,
wherein the distal end of the inflatable balloon attaches to the distal end of the catheter shaft and wherein the proximal end of the inflatable balloon attaches to the catheter shaft at a distance from the distal end of the balloon that is less than the unfolded longitudinal dimension of the inflatable balloon, and
inflating the inflatable balloon, whereby a distal region of the inflatable balloon moves distally to at least the distal end of the catheter shaft and presses against the occluded portion of the vessel and against a wall of the vessel immediately adjacent to the occluded portion.

16. The method of claim 15, wherein the distal region of the inflatable balloon moves distally beyond the distal end of the catheter shaft.

17. The method of claim 15, wherein the vessel is a vessel of the urinary system.

18. The method of claim 17, wherein the occlusion results from the presence of a kidney stone.

19. The method of claim 18, wherein a portion of the vessel directly below the kidney stone is expanded upon inflation of the inflatable balloon.
